(19) United States
(12) Patent Application Publication
(10) Pub. No.: US 2010/0022951 A1
(43) Pub. Date: Jan. 28, 2010

(54) DETACHABLE HUB/LUER DEVICE AND PROCESSES

(75) Inventors: David A. Ferrera, Redondo Beach, CA (US); John Fulkerson, Irvine, CA (US)

Correspondence Address: Luce, Forward, Hamilton & Scripps LLP, 2050 Main Street, Suite 600, Irvine, CA, 92614, US

(73) Assignee: LUCE, FORWARD, HAMILTON 7 SCRIPPS, LLP, Irvine, CA (US)

(21) Appl. No.: 12/469,462

(22) Filed: May 20, 2009

Related U.S. Application Data
(63) Continuation-in-part of application No. 12/123,390, filed on May 19, 2008.
Application No. 12/123,390 is a continuation-in-part of application No. 12/422,105, filed on Apr. 10, 2009.
(60) Provisional application No. 61/057,613, filed on May 30, 2008.
(60) Provisional application No. 61/166,725, filed on Apr. 4, 2009.

(51) Int. Cl. A61M 25/18; B23P 17/04

(52) U.S. Cl. 604/103; 604/533; 623/1.11; 29/428

(57) ABSTRACT

A hub system comprising, in combination: a luer further comprising a luer channel, configured to accommodate a selected device, said luer channel further comprising a segment extending through an extension of said luer; and a nose further comprising a nose channel configured to selectively couple with said extension, wherein the inner diameter of at least a portion of said nose channel is less than the outer diameter of the extension. A method of using the same to secure a device within said system.

### RELATED APPLICATIONS

This application claims the Paris Convention Priority of: U.S. Provisional Application Ser. No. 61/057,613, filed on May 30, 2008; U.S. Provisional Application Ser. No. 61/166,725, filed Apr. 4, 2009; is a continuation-in-part of U.S. Utility application Ser. No. 12/123,390, filed on May 19, 2008; and is a continuation-in-part of U.S. Utility application Ser. No. 12/422,105, filed Apr. 10, 2009 by the present and the instant assignee, each of which is incorporated by reference as if fully set forth herein.

### BACKGROUND OF THE INVENTION

1. Field

This disclosure relates to systems and methods for addressing, assessing, and/or treating vascular issues. Specifically, this disclosure relates to systems and methods for treating ischemic stroke or other vascular issues caused by an occlusion within blood vessels.

2. General Background

Devices for treating vascular issues, such as those utilizing catheters, are useful for addressing issues of the neurological and coronary vasculature. Assemblies may be formed to facilitate operation of the devices by a medical professional during endovascular procedures. Variations in the assemblies provide a variety of functionalities to a surgeon manipulating such devices, which have become a standard of treatment for cutting edge specialists in several related disciplines.

### SUMMARY OF THE INVENTION

Briefly stated, where two or more components of an assembly may be joined, the manner in which they are joined and the connection between them substantially impacts affect the ease, efficiency, and speed with which the assembly and associated devices may be used. In situations where immediate flow or degrees of flow are mission critical, such devices are of primary significance.

According to embodiments of the present disclosure, a hub system is disclosed. According to embodiments, the hub system comprises, in combination: a luer further comprising a luer channel, configured to accommodate a selected device, the luer channel further comprising a segment extending through an extension of the luer; and a nose further comprising a nose channel configured to selectively couple with the extension, wherein the nose channel is configured to compress the extension when the nose channel is coupled to the extension. The hub system may further comprise a gasket configured to fit between at least a portion of the luer and at least a portion of the nose.

The luer channel may connect a proximal opening disposed at a proximal end of the luer and a distal opening disposed at a distal end of the luer. The luer channel may be substantially tapered from the proximal opening to the segment.

The extension may be compressible. The segment may be configured to have a compressed state and an uncompressed state, corresponding to the state of the extension. The segment may be configured to allow travel of the selected device while in an uncompressed state and configured to restrict travel of the selected device while in a compressed state.

The luer may further comprise a luer interface configured to interface with a nose interface of the nose and configured to facilitate coupling of the luer to the nose. The nose further may further comprise a nose interface configured to interface with a luer interface of the luer and configured to facilitate coupling of the nose to the luer.

According to embodiments of the present disclosure, a method of use is disclosed. According to embodiments, the method comprises, in combination: placing a selected device within a luer channel of a luer, including within a segment of the luer channel; placing the selected device within a gasket; placing the selected device within a nose channel of a nose, such that the luer and the nose are substantially aligned along the length of the selected device, with a distal end of the luer facing a proximal end of the nose, and with the gasket between the luer and the nose; advancing the selected device through the luer and the nose to a desired location; and coupling the nose to the luer, whereby mobility of the selected device is restricted within at least one of the luer and the nose by compressing the segment onto a at least a portion of the selected device.

The desired location may correspond to a location inside an anatomy of a body. Coupling the nose to the luer may include advancing the extension to within the nose channel through a proximal opening of the nose; causing the distal end of the luer to overlap with the proximal end of the nose; or operating a luer interface relative to a nose interface. The method may further comprise de-coupling the nose from the luer, whereby mobility of the selected device may be restored.

According to embodiments of the present disclosure, a kit is disclosed. According to embodiments, the kit comprises, in combination: a luer further comprising a luer channel, the luer channel further comprising a segment extending through an extension of the luer; a nose further comprising a nose channel configured to selectively couple with the extension, wherein the inner diameter of at least a portion of the nose channel is less than the outer diameter of the extension, such that the nose channel is configured to compress the extension when the nose channel is coupled to the extension; a gasket configured to fit between at least a portion of the luer and at least a portion of the nose; and instructions for use.

The kit may further comprise a selected device configured to be advanced within at least one of the luer channel and the nose channel. The selected device may include at least one of a guidewire, an over-the-wire or rapid exchange catheter, a microcatheter, a balloon, a stent, a coil, and a device for vascular treatment.

### BRIEF DESCRIPTION OF THE FIGURES

The above-mentioned features and objects of the present disclosure will become more apparent with reference to the following description taken in conjunction with the accompanying drawings wherein like reference numerals denote like elements and in which:

FIG. 1 shows a plan view of a luer, according to embodiments of the present disclosure;

FIG. 2 shows a plan view of a luer, according to embodiments of the present disclosure;

FIG. 3 shows a side view of a luer, according to embodiments of the present disclosure;

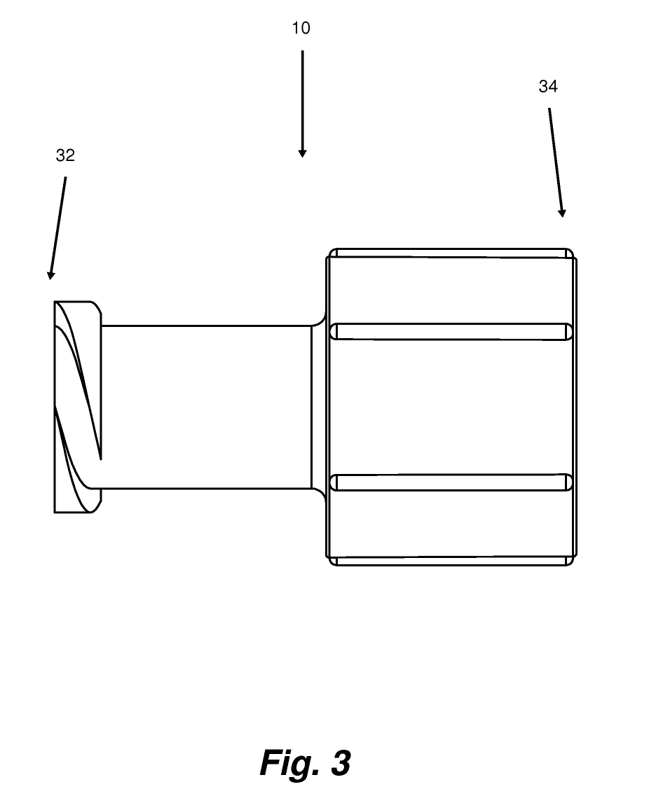

FIG. 4 shows a mid-sagittal/cross-sectional view of a luer, according to embodiments of the present disclosure;

FIG. 5 shows a view of a proximal end of a luer, according to embodiments of the present disclosure;

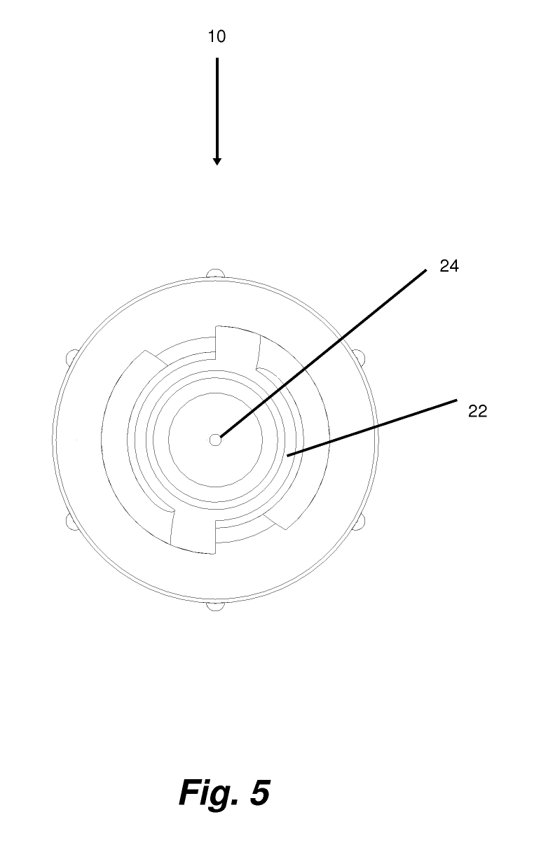

FIG. 6 shows a view of a distal end of a luer, according to embodiments of the present disclosure;

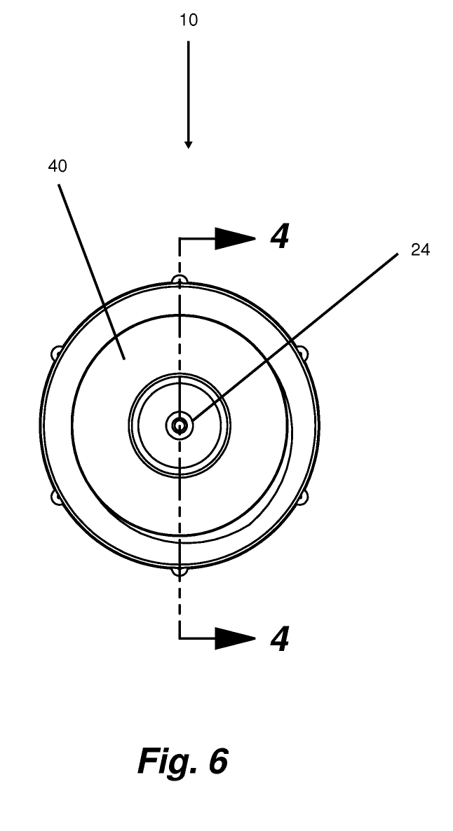

FIG. 7 shows a plan view of a nose, according to embodiments of the present disclosure;

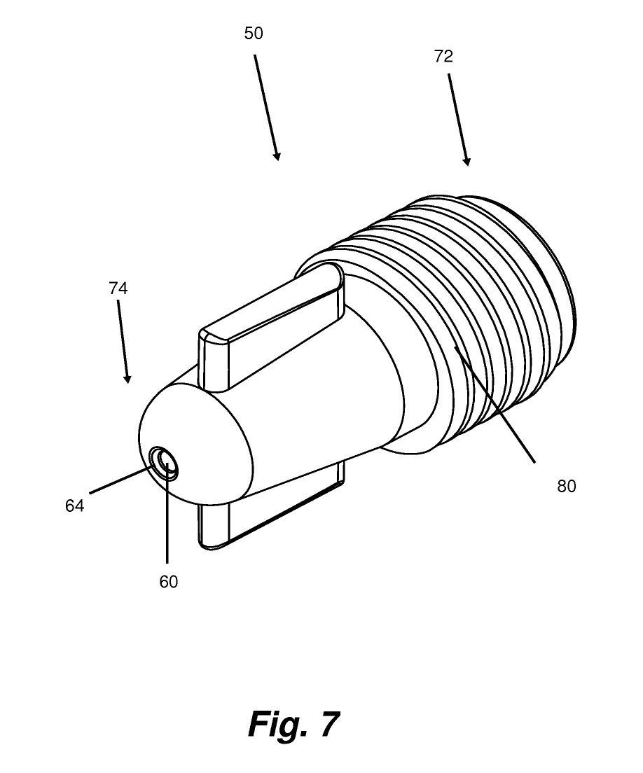

FIG. 8 shows a side view of a nose, according to embodiments of the present disclosure;

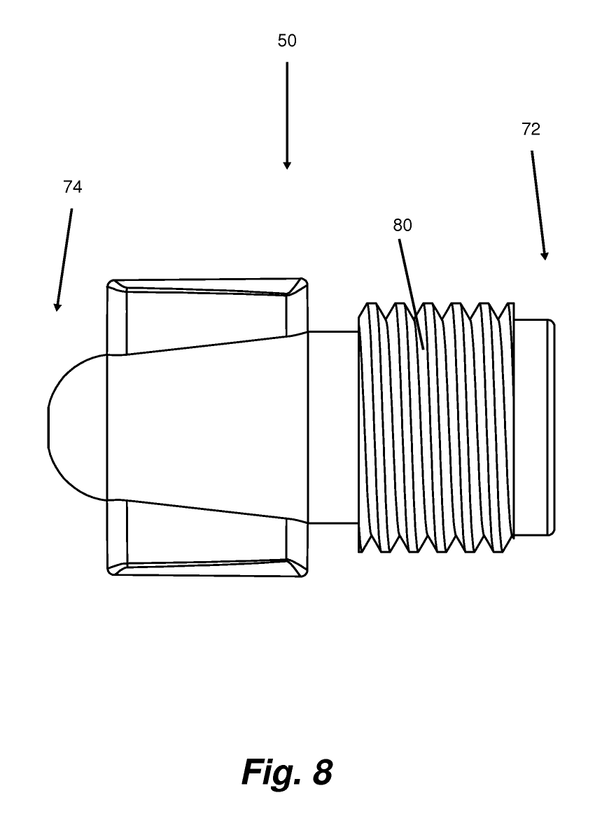

FIG. 9 shows a mid-sagittal/cross-sectional view of a nose, according to embodiments of the present disclosure;

FIG. 10 shows a view of a distal end of a nose, according to embodiments of the present disclosure;

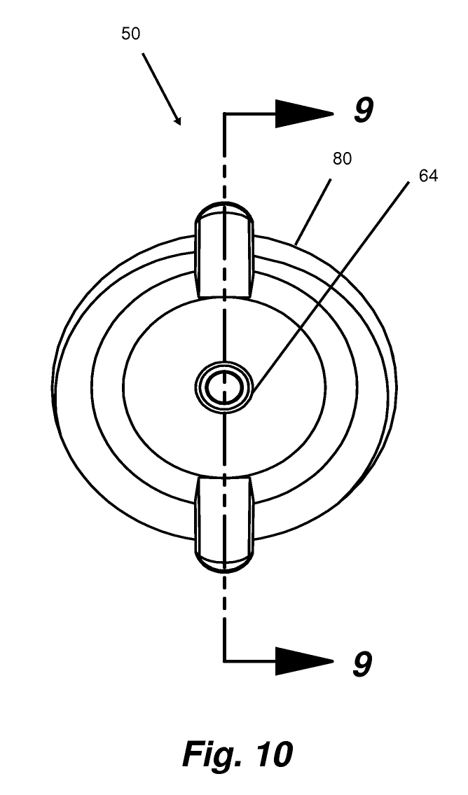

FIG. 11 shows a view of a proximal end of a nose, according to embodiments of the present disclosure;

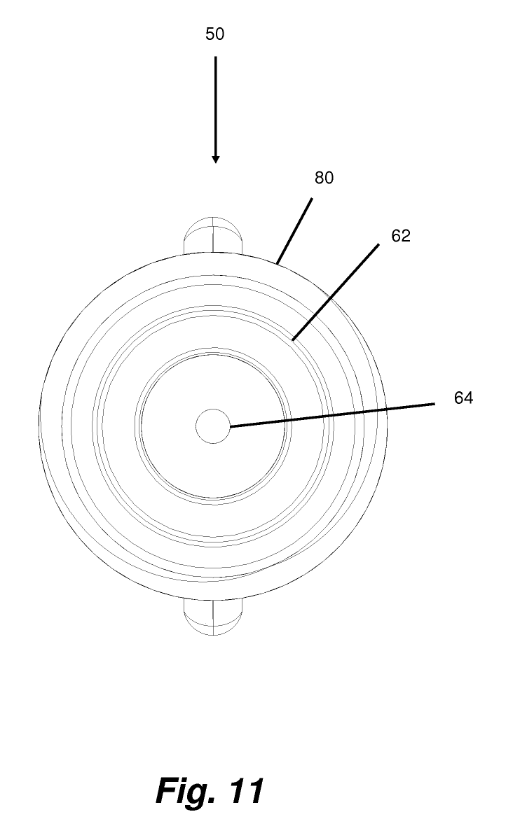

FIG. 12 shows a plan view of a luer and a nose, according to embodiments of the present disclosure; and

FIG. 13 shows a side view of a nose, a gasket, and a luer, according to embodiments of the present disclosure.

### DETAILED DESCRIPTION

According to embodiments, a hub system comprising luer 10 and nose 50 is disclosed. Luer 10 may include a proximal opening 22 disposed at proximal end 32 of luer 10 and distal opening 24 disposed at or near distal end 34 of luer 10. Luer channel 20 may connect proximal opening 22 with distal opening 24.

According to embodiments, luer channel 20 may be configured to house a catheter device or a device configured to be disposed within a catheter. For example, luer channel 20 of luer 10 may be configured to accommodate guidewires, over-the-wire or rapid exchange catheters, microcatheters, balloons, stents, coils, or devices for other vascular treatment, such as devices for addressing stroke, embolism, aneurysm, or coronary issues.

According to embodiments, proximal opening 22 may be configured to receive a device for entry into luer channel 20. Distal opening 24 may be configured to direct a device passing through luer channel 20, such that the direction of the device exiting the luer channel 20 through distal opening 24 is guided by the geometry of distal opening 24 and its position relative to luer channel 20.

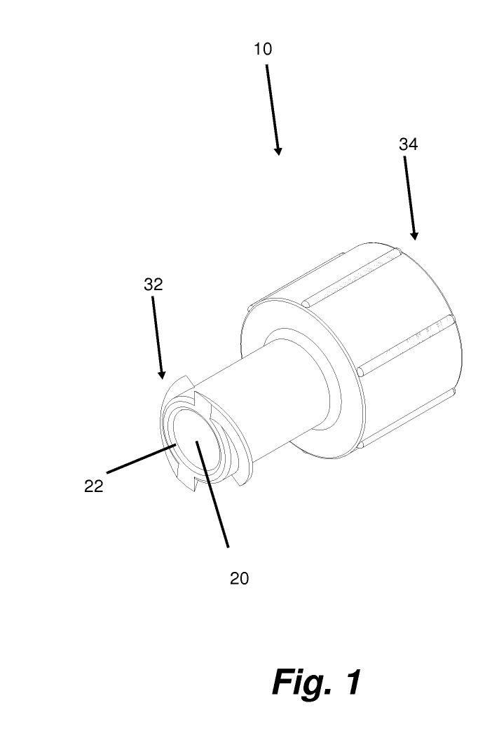

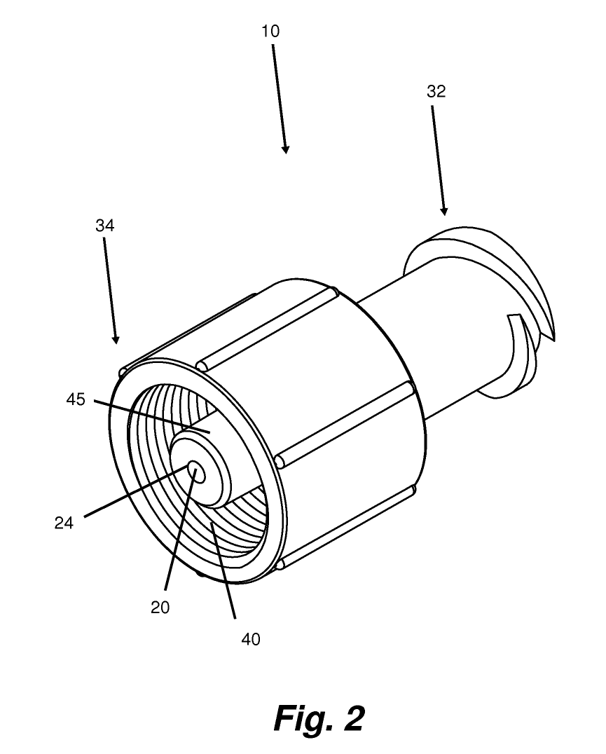

According to embodiments, proximal opening 22 may be at least as large as distal opening 24. For example, proximal opening 22 may have a greater diameter than distal opening 24, as shown in FIGS. 1 and 2. Proximal opening 22 may be configured to receive a device entering luer channel 20 at a variety of angles relative to an axis extending from proximal opening 22 to distal opening 24.

According to embodiments, at least one of proximal end 32 and distal end 34 are configured to connect to other devices. For example, luer fittings and/or attachments may be provided at proximal end 32 or distal end 34. The fittings and attachments may be provided according to known standards generally accepted in the field, providing compatibility with a number of related instruments. For example, luer 10 may be configured to interface with a Touhy Borst adapter, a catheter having a lumen, a flush line, a rotating hemostatic valve (RHV), or other selected device for use in combination with a catheter device. According to embodiments, luer channel 20 may provide a seamless transition to any device attached at proximal end 32 and/or distal end 34.

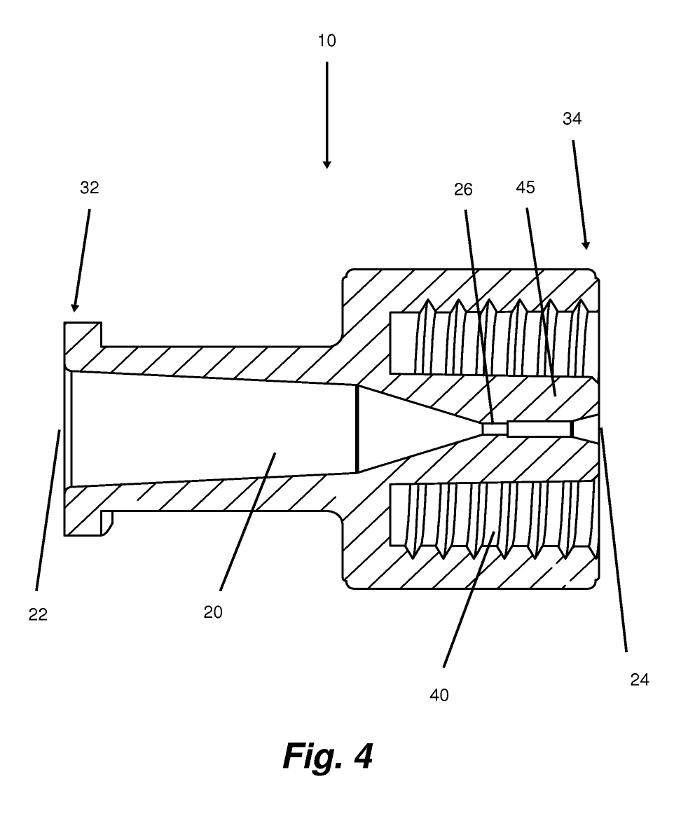

According to embodiments, luer channel 20 may have one of a variety of shapes or geometries to provide improved connection between proximal opening 22 and distal opening 24. According to embodiments, luer channel 20 may be generally tapered from a portion near proximal opening 22 to a portion near distal opening 24, such that a smooth transition from proximal opening 22 to distal opening 24 is provided for a device within luer channel 20. For example, at least a portion of luer channel 20 may be at least substantially linearly tapered, as shown in FIG. 4. At least a portion of luer channel 20 may be tapered such that a cross section shows a parabola or other substantially curve-linear shape. At least a portion of luer channel 20 may be tapered such that paths along the walls of luer channel 20 from proximal end 32 to distal end 34 contain no “steps”, discontinuities, or disjunctions.

According to embodiments, luer 10 having luer channel 20 with tapered geometry may be made by using a bore having complementary tapered geometry. According to embodiments, luer 10 having luer channel 20 with tapered geometry may be made with a mold having a shape that yield the desired tapered characteristics. Those skilled in the art will understand that other methods of making luer 10 may be contemplated in light of the present disclosure.

According to embodiments, luer channel 20 of luer 10 may comprise a segment 26. As shown in FIG. 4, segment 26 may be a portion of luer channel 20 that has a small cross sectional diameter relative to other portions of luer channel 20. According to embodiments, segment 26 may be a portion of luer channel 20 that has a small cross sectional diameter relative to other portions of luer channel 20 near luer interface 40.

According to embodiments, segment 26 corresponds to a portion of luer 10 near distal end 34. For example, segment 26 may be within extension 45 of luer 10, as shown in FIG. 4. Extension 45 may be at least radially compressible, such that the size, shape, and geometry of segment 26 is affected by the radial compression of extension 45. Segment 26, in an uncompressed state, may have such a size and geometry as to accommodate a device within luer channel 20. For example, the diameter of segment 26, in an uncompressed state, may be at least greater than that of a device within luer channel 20. In a compressed state, the diameter of segment 26 may be equal to or less than that of a device within luer channel 20.

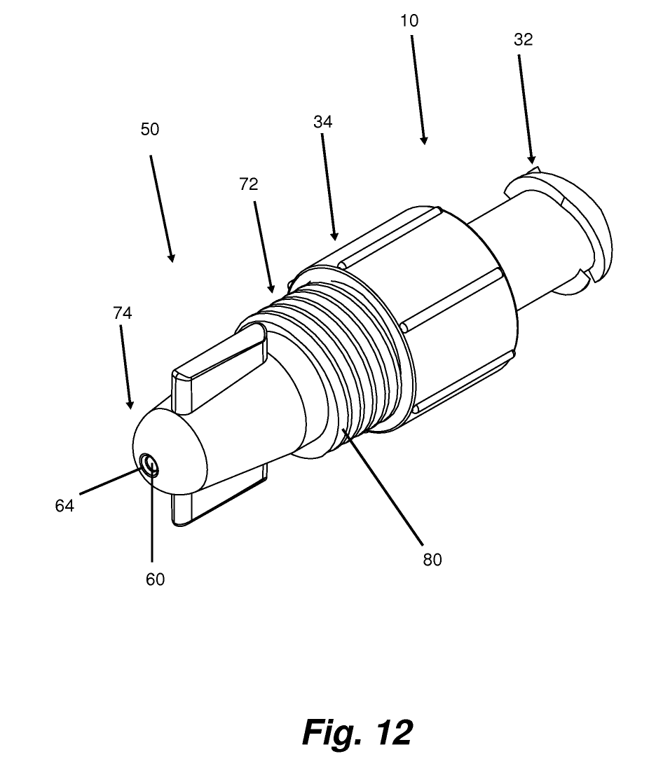

According to embodiments, luer 10 may comprise luer interface 40. Luer interface 40 may be configured to receive and connect with nose interface 80 of nose 50, as shown in FIG. 12 and as disclosed further herein. Luer interface 40 may be within a recessed female portion of luer 10, as shown in FIG. 4, or may be on an extended male portion (not shown).

According to embodiments, an improved nose 50 is disclosed. Nose 50 may include a proximal opening 62 disposed at proximal end 72 of nose 50 and distal opening 64 disposed at or near distal end 74 of nose 50. Nose channel 60 may connect proximal opening 62 with distal opening 64.

According to embodiments, nose channel 60 may be configured to house a catheter device or a device configured to be disposed within a catheter. For example, nose channel 60 of nose 50 may be configured to accommodate guidewires, over-the-wire or rapid exchange catheters, microcatheters, balloons, stents, coils, or devices for other vascular treatment, such as devices for addressing stroke, embolism, aneurysm, or coronary issues.

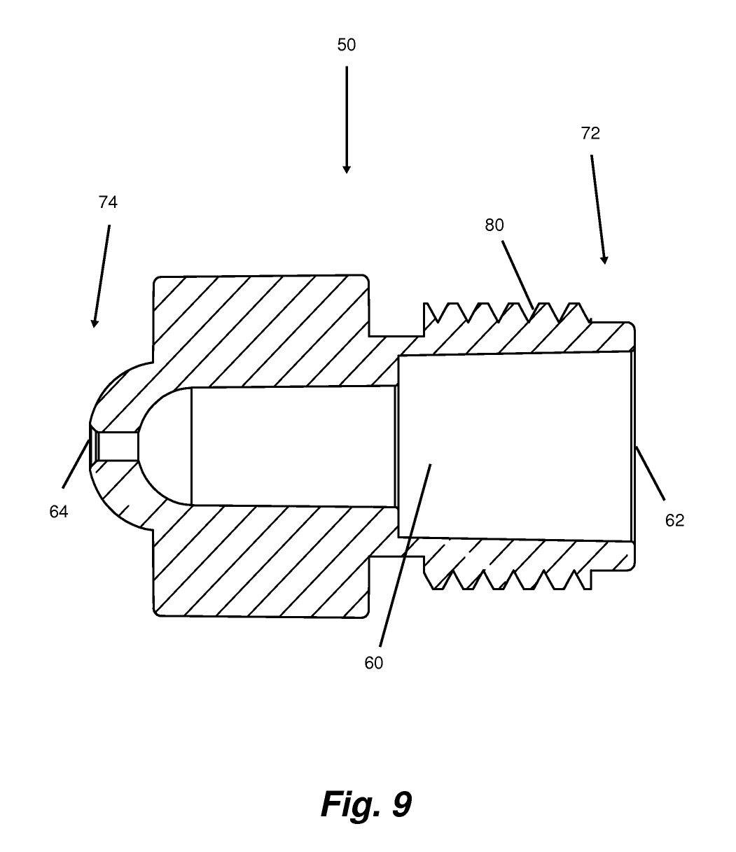

According to embodiments, nose 50 includes nose interface 80. Nose interface 80 may be configured to receive and connect with luer interface 40 of luer 10, as shown in FIG. 12 and as disclosed further herein. Nose interface 80 may be on an extended male portion, as shown in FIG. 9, or may be within a recessed female portion of luer 10 (not shown).

According to embodiments, at least a portion of nose channel 60 of nose 50 is configured to encompass and circumscribe at least a portion of luer 10. For example, extension 45 of luer 10 may be configured to be encompassed by nose channel 60 of nose 50. The portion of luer 10 encompassed by the portion of nose channel 60 may correspond to the portion of luer 10 where segment 26 is located. For example, proximal end 72 of nose 50 may be caused to overlap with distal end 34 of luer 10, such that a portion of luer 10 near distal end 34 is within a portion of nose channel 60 near proximal end 72 of nose 50.

According to embodiments, as the amount of overlap between proximal end 72 of nose 50 and distal end 34 of luer 10 increases, extension 45 of luer 10 is compressed by nose channel 60 of nose 50. For example, as shown in FIG. 9, nose channel 60 may have tapered geometry, such that the diameter of nose channel 60 decreases along nose channel 60 from proximal opening 62 toward distal opening 64. Other configurations of nose channel 60 for compressing extension 45 are contemplated, including notches, protrusions, rings, and extensions extending inward from the walls of nose channel 60. Where at least radial compression of extension 45 reduces the cross-sectional size (i.e., diameter) of segment 26 of luer channel 20, segment 26 may restrict a device within segment 26, such that the device is secured. Compression of extension 45 may have other desired effects, such as restriction of fluid flow through segment 26 or through a device within segment 26. A device within segment 26 may be released by enlargement of segment 26, effectuated by expansion of extension 45. Such expansion may be effectuated by removal of extension 45 from within nose channel 60 of nose 50.

According to embodiments, luer interface 40 and nose interface 80 may be configured to facilitate the overlap of proximal end 72 of nose 50 and distal end 34 of luer 10. For example, as shown in FIGS. 4 and 9, luer interface 40 and nose interface 80 may include complementary threading, such that rotation of one of luer 10 and nose 50 relative to the other increases or decreases the amount of overlap, depending on the direction of rotation. According to embodiments, nose 50 may be rotated relative to luer 10 such that a device within luer 10 is not subjected to torque. According to embodiments, other configurations for luer interface 40 and nose interface 80 are contemplated, including locks, snaps, ratchets, and fittings, including those accepted as compliant with standards within a relevant industry.

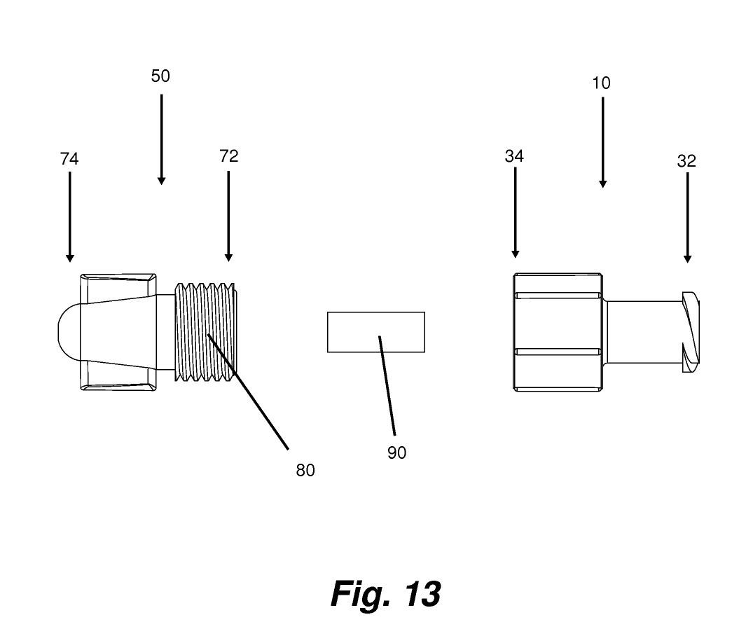

According to embodiments, a hub device further comprises a gasket 90. As shown in FIG. 13, gasket 90 may be configured to fit between at least a portion of luer 10 and at least a portion of nose 50. For example, gasket 90 may be configured to fit around the outer walls of extension 45. Gasket 90 may be configured to fit within the inner walls of nose channel 60. According to embodiments, gasket 90 facilitates coupling of luer 10 and nose 50 and provides compression to at least a portion of luer 10. Such compression may be communicated to at least segment 26 of luer channel 20, whereby a selected device within luer channel 20 is secured. Gasket 90 may be tapered to facilitate compression. The geometry of gasket 90 may cause the extent of compression to be variable according to the extent of overlap between luer 10 and nose 50. Gasket 90 may include silicone or at least other compressible materials.

According to embodiments, a method of using a hub system comprising luer 10 and nose 50 is disclosed. According to embodiments, a device is placed within luer channel 20 of luer 10, including within segment 26. Such a device may include guidewires, over-the-wire or rapid exchange catheters, microcatheters, balloons, stents, coils, or devices for other vascular treatment, such as devices for addressing stroke, embolism, aneurysm, or coronary issues. The device may also be placed within nose channel 60 of nose 50, such that both luer 10 and nose 50 are aligned along the length of the device, with distal end 34 of luer 10 be substantial near or adjacent to proximal end 72 of nose 50. According to embodiments, where gasket 90 is used, the device may also be placed within gasket 90 such that gasket 90 is disposed between luer 10 and nose 50, such that gasket 90 is positioned to facilitate coupling of luer 10 and nose 50.

According to embodiments, the device is advanced through luer 10 and nose 50 to desired location. Such a location may correspond to a location inside the anatomy of a body. When advancement or retraction of the device relative to either one of luer 10 and nose 50 is no longer desired, nose 50 may be coupled to luer 10.

According to embodiments, nose 50 is coupled to luer 10 by bringing extension 45 of luer 10 within nose channel 60 of nose 50 via proximal opening 62, whereby overlap of distal end 34 of luer 10 and proximal end 72 of nose 50 is increased. According to embodiments, luer interface 40 or nose interface 80 may facilitate advancement of nose 50 relative to luer 10. For example, threading may allow a user to rotate nose 50 onto luer 10.

According to embodiments, nose channel 60 of nose 50 at least radially compresses extension 45 as nose 50 is coupled to luer 10. As a result, segment 26 of luer channel 20 is compressed, and pressure or friction is applied to the device within segment 26.

According to embodiments, a kit is disclosed. The kit may comprise luer 10, nose 50, gasket 90, and instructions for use. The kit may further comprise at least one device configured to interface with proximal end 32 of luer 10. The kit may further comprise at least one device configured to be housed within luer channel 20 of luer 10 or nose channel 60 of nose 50, as further disclosed with respect to embodiments disclosed herein. Likewise, directions for use (“DFU”) are included and the device may be part of a surgical tray or other packaged accessory set for surgeries. The kit may be a sub-component of a surgical tray.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A hub system comprising, in combination:
a luer further comprising a luer channel, configured to accommodate a selected device, said luer channel further comprising a segment extending through an extension of said luer; and
a nose further comprising a nose channel configured to selectively couple with said extension, wherein said nose channel is configured to compress said extension when said nose channel is coupled to said extension.

2. The hub system of claim 1, further comprising a gasket configured to fit between at least a portion of said luer and at least a portion of said nose.

3. The hub system of claim 1, wherein said luer channel connects a proximal opening disposed at a proximal end of said luer and a distal opening disposed at a distal end of said luer.

4. The hub system of claim 3, wherein said luer channel is substantially tapered from said proximal opening to said segment.

5. The hub system of claim 1, wherein said extension is compressible.

6. The hub system of claim 1, wherein said segment is configured to have a compressed state and an uncompressed state, corresponding to a state of said extension.

7. The hub system of claim 6, wherein said segment is configured to allow travel of said selected device while in said uncompressed state.

8. The hub system of claim 6, wherein said segment is configured to restrict travel of said selected device while in said compressed state.

9. The hub system of claim 1, wherein said selected device includes at least one of a guidewire, an over-the-wire or rapid exchange catheter, a microcatheter, a balloon, a stent, a coil, and a device for vascular treatment.

10. The hub system of claim 1, wherein said luer further comprises a luer interface configured to interface with a nose interface of said nose and configured to facilitate coupling of said luer to said nose.

11. The hub system of claim 1, wherein said nose further comprises a nose interface configured to interface with a luer interface of said luer and configured to facilitate coupling of said nose to said luer.

12. A method, comprising, in combination:
placing a selected device within a luer channel of a luer, including within a segment of said luer channel;
placing said selected device within a gasket;
placing said selected device within a nose channel of a nose, such that said luer and said nose are substantially aligned along a length of the selected device, with a distal end of said luer facing a proximal end of said nose, and with said gasket disposed between said luer and said nose;
advancing said selected device through said luer and said nose to a desired location; and
coupling said nose to said luer, whereby mobility of said selected device is restricted within at least one of said luer and said nose by compressing said segment onto a at least a portion of said selected device.

13. The method of claim 12, wherein said desired location corresponds to a location inside an anatomy of a body.

14. The method of claim 12, wherein coupling said nose to said luer includes advancing an extension within said nose channel through a proximal opening of said nose.

15. The method of claim 12, wherein coupling said nose to said luer includes causing said distal end of said luer to overlap with said proximal end of said nose.

16. The method of claim 12, wherein coupling said nose to said luer includes operating a luer interface relative to a nose interface.

17. The method of claim 12, further comprising de-coupling said nose from said luer, whereby mobility of said selected device is restored.

18. A kit, comprising, in combination:
a luer further comprising a luer channel, said luer channel further comprising a segment extending through an extension of said luer;
a nose further comprising a nose channel configured to selectively couple with said extension, wherein an inner diameter of at least a portion of said nose channel is less than an outer diameter of said extension, such that said nose channel is configured to compress said extension when said nose channel is coupled to said extension;
a gasket configured to fit between at least a portion of said luer and at least a portion of said nose; and
instructions for use.

19. The kit of claim 18, further comprising a selected device configured to be advanced within at least one of said luer channel and said nose channel.

20. The kit of claim 18, wherein a selected device includes at least one of a guidewire, an over-the-wire or rapid exchange catheter, a microcatheter, a balloon, a stent, a coil, and a device for vascular treatment.
